(19) United States
(12) Patent Application Publication
(10) Pub. No.: US 2025/0245705 A1
(43) Pub. Date: Jul. 31, 2025

(54) Charitable Funding System and Method

(71) Applicant: Qualify Health, Inc., Palm Beach Gardens, FL (US)

(72) Inventors: Monique Lappas, Palm Beach Gardens, FL (US); Jennifer M. Borland, Port Saint Lucie, FL (US); Kelly M. Sanders, Stuart, FL (US)

(21) Appl. No.: 19/036,769

(22) Filed: Jan. 24, 2025

Related U.S. Application Data
(60) Provisional application No. 63/625,115, filed on Jan. 25, 2024.

(51) Int. Cl. G06Q 30/0279

(52) U.S. Cl. CPC G06Q 30/0279

(57) ABSTRACT

A method, computer program product, and computing system for identifying one or more outstanding medical bills associated with one or more patients; identifying one or more charitable organizations available to at least partially satisfy the one or more outstanding medical bills; and applying the one or more patients for funding via the one or more charitable organizations.

### PRIORITY APPLICATION

This application claims the benefit of U.S. Provisional Application No. 63/625,115, filed on 25 Jan. 2024, the entire contents of which are herein incorporated by reference.

### TECHNICAL FIELD

This disclosure relates to charitable funds and, more particularly, to systems and methods for matching those charitable funds to people in need.

### BACKGROUND

The history of charitable funds dedicated to covering medical expenses is rooted in a long tradition of community solidarity and philanthropy. In ancient times, religious institutions like temples, monasteries, and churches played a significant role in caring for the sick, funded through donations from the community. Many religious traditions emphasized the importance of providing for the ill, leading to the establishment of hospitals and care centers. During the Enlightenment and Industrial Revolution, secular charitable organizations began addressing public health needs, often supported by wealthy individuals and philanthropists. The 19th century saw the rise of organizations like the Red Cross, which provided systematic aid to those in need. By the 20th century, charities had evolved to fill gaps left by public healthcare systems, focusing on specific diseases, emergency medical care, or vulnerable populations. Today, charitable funds range from global organizations addressing widespread healthcare issues to smaller local initiatives targeting specific needs. Modern platforms like GoFundMe have also emerged, enabling individuals to raise money for medical expenses, expanding the concept of medical charity.

Charitable funds are used to cover a wide array of medical expenses, including direct costs like surgeries, hospital stays, medications, and specialized treatments, as well as indirect costs such as travel for care, lodging near treatment facilities, and lost income. Many charities also provide support services like counseling, rehabilitation, or palliative care, and some focus on preventative measures such as vaccinations and health education. Emergency relief funds help individuals affected by disasters, conflicts, or pandemics, while others fund medical research or infrastructure in underserved areas.

Despite their significant impact, accessing these funds can be challenging. Many people are unaware of available resources or how to locate them, as charitable funds are often scattered across various organizations with limited visibility. Eligibility criteria can be complex, requiring applicants to meet specific income thresholds, medical conditions, or geographic restrictions. Navigating these requirements often involves extensive documentation and can be overwhelming. Additionally, the fragmented nature of the charitable sector and the lack of a centralized directory make it difficult to identify relevant funds. Many organizations operate on limited budgets, which can lead to caps on assistance or long waiting lists. Regional disparities also create challenges, as individuals in rural or underserved areas may have less access to charitable support.

### Summary of Disclosure

### Concept 4-Reactively Processing Outstanding Bills

In one implementation, a computer-implemented method is executed on a computing device and includes: identifying one or more outstanding medical bills associated with one or more patients; identifying one or more charitable organizations available to at least partially satisfy the one or more outstanding medical bills; and applying the one or more patients for funding via the one or more charitable organizations.

One or more of the following features may be included. The one or more charitable organizations may include one or more of: a charity; a non-profit organization and a philanthropic entity of a for-profit entity. The one or more charitable organizations may be defined within a charitable organization database. The funding may include one or more of: drug-based funding; treatment-based funding; and disease-based funding. Applying the one or more patients for funding via the one or more charitable organizations may include: automatically applying the one or more patients for funding via an electronic application process. Funding may be obtained from the one or more charitable organizations for the one or more outstanding medical bills associated with one or more patients. The obtained funds may be applied to at least partially satisfy the one or more outstanding medical bills associated with one or more patients. A charitable organization database may be defined that defines application criteria for a plurality of charitable organizations. Applying the one or more patients for funding via the one or more charitable organizations may include: populating an application for the one or more patients for the one or more charitable organizations, thus defining one or more populated applications. Applying the one or more patients for funding via the one or more charitable organizations further may include: submitting the one or more populated applications to the one or more charitable organizations.

In another implementation, a computer program product resides on a computer readable medium and has a plurality of instructions stored on it. When executed by a processor, the instructions cause the processor to perform operations including: identifying one or more outstanding medical bills associated with one or more patients; identifying one or more charitable organizations available to at least partially satisfy the one or more outstanding medical bills; and applying the one or more patients for funding via the one or more charitable organizations.

One or more of the following features may be included. The one or more charitable organizations may include one or more of: a charity; a non-profit organization and a philanthropic entity of a for-profit entity. The one or more charitable organizations may be defined within a charitable organization database. The funding may include one or more of: drug-based funding; treatment-based funding; and disease-based funding. Applying the one or more patients for funding via the one or more charitable organizations may include: automatically applying the one or more patients for funding via an electronic application process. Funding may be obtained from the one or more charitable organizations for the one or more outstanding medical bills associated with one or more patients. The obtained funds may be applied to at least partially satisfy the one or more outstanding medical bills associated with one or more patients. A charitable organization database may be defined that defines application criteria for a plurality of charitable organizations. Applying the one or more patients for funding via the one or more charitable organizations may include: populating an application for the one or more patients for the one or more charitable organizations, thus defining one or more populated applications. Applying the one or more patients for funding via the one or more charitable organizations further may include: submitting the one or more populated applications to the one or more charitable organizations.

In another implementation, a computing system including a processor and memory is configured to perform operations including: identifying one or more outstanding medical bills associated with one or more patients; identifying one or more charitable organizations available to at least partially satisfy the one or more outstanding medical bills; and applying the one or more patients for funding via the one or more charitable organizations.

One or more of the following features may be included. The one or more charitable organizations may include one or more of: a charity; a non-profit organization and a philanthropic entity of a for-profit entity. The one or more charitable organizations may be defined within a charitable organization database. The funding may include one or more of: drug-based funding; treatment-based funding; and disease-based funding. Applying the one or more patients for funding via the one or more charitable organizations may include: automatically applying the one or more patients for funding via an electronic application process. Funding may be obtained from the one or more charitable organizations for the one or more outstanding medical bills associated with one or more patients. The obtained funds may be applied to at least partially satisfy the one or more outstanding medical bills associated with one or more patients. A charitable organization database may be defined that defines application criteria for a plurality of charitable organizations. Applying the one or more patients for funding via the one or more charitable organizations may include: populating an application for the one or more patients for the one or more charitable organizations, thus defining one or more populated applications. Applying the one or more patients for funding via the one or more charitable organizations further may include: submitting the one or more populated applications to the one or more charitable organizations.

The details of one or more implementations are set forth in the accompanying drawings and the description below. Other features and advantages will become apparent from the description, the drawings, and the claims.

### BRIEF DESCRIPTION OF THE DRAWINGS

FIG. 1 is a diagrammatic view of a distributed computing network including a computing device that executes a charitable funding process according to an implementation of the present disclosure;

FIG. 2 is a diagrammatic view of a claim submissions instance effectuated by the charitable funding process of FIG. 1 according to an implementation of the present disclosure;

FIG. 3 is a flowchart of an implementation of the charitable funding process of FIG. 1 according to an implementation of the present disclosure;

FIG. 4 is a flowchart of another implementation of the charitable funding process of FIG. 1 according to an implementation of the present disclosure;

FIG. 5 is a flowchart of another implementation of the charitable funding process of FIG. 1 according to an implementation of the present disclosure;

FIG. 6 is a flowchart of another implementation of the charitable funding process of FIG. 1 according to an implementation of the present disclosure;

FIG. 7 is a flowchart of another implementation of the charitable funding process of FIG. 1 according to an implementation of the present disclosure; and

FIG. 8 is a flowchart of another implementation of the charitable funding process of FIG. 1 according to an implementation of the present disclosure.

Like reference symbols in the various drawings indicate like elements.

### DETAILED DESCRIPTION OF THE PREFERRED EMBODIMENTS

### System Overview:

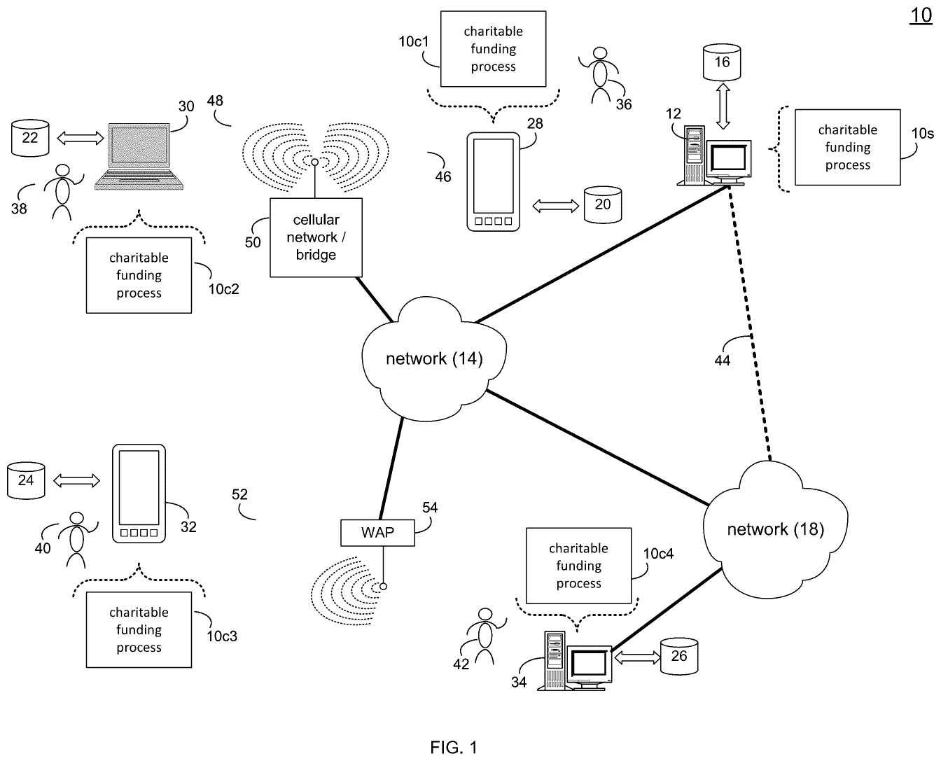

Referring to FIG. 1, there is shown charitable funding process 10. As will be discussed below in greater detail, charitable funding process 10 may be configured to match people in need of assistance with paying their medical expenses with charitable funds that offer such assistance.

Charitable funding process 10 may be implemented as a server-side process, a client-side process, or a hybrid server-side/client-side process. For example, charitable funding process 10 may be implemented as a purely server-side process via charitable funding process 10s. Alternatively, charitable funding process 10 may be implemented as a purely client-side process via one or more of charitable funding process 10c1, charitable funding process 10c2, charitable funding process 10c3, and charitable funding process 10c4. Alternatively still, charitable funding process 10 may be implemented as a hybrid server-side/client-side process via charitable funding process 10s in combination with one or more of charitable funding process 10c1, charitable funding process 10c2, charitable funding process 10c3, and charitable funding process 10c4. Accordingly, charitable funding process 10 as used in this disclosure may include any combination of charitable funding process 10s, charitable funding process 10c1, charitable funding process 10c2, charitable funding process 10c3, and charitable funding process 10c4.

Charitable funding process 10s may be a server application and may reside on and may be executed by computing device 12, which may be connected to network 14 (e.g., the Internet or a local area network). Examples of computing device 12 may include, but are not limited to: a personal computer, a laptop computer, a personal digital assistant, a data-enabled cellular telephone, a notebook computer, a television with one or more processors embedded therein or coupled thereto, a cable/satellite receiver with one or more processors embedded therein or coupled thereto, a server computer, a series of server computers, a mini computer, a mainframe computer, or a dedicated network device.

The instruction sets and subroutines of charitable funding process 10s, which may be stored on storage device 16 coupled to computing device 12, may be executed by one or more processors (not shown) and one or more memory architectures (not shown) included within computing device 12. Examples of storage device 16 may include but are not limited to: a hard disk drive; a tape drive; an optical drive; a RAID device; a random access memory (RAM); a read-only memory (ROM); and all forms of flash memory storage devices.

Network 14 may be connected to one or more secondary networks (e.g., network 18), examples of which may include but are not limited to: a local area network; a wide area network; or an intranet, for example.

Examples of charitable funding processes 10c1, 10c2, 10c3, 10c4 may include but are not limited to a web browser, a game console user interface, or a specialized application (e.g., an application running on e.g., the Android™ platform or the iPhone™ platform). The instruction sets and subroutines of roadside assistance applications 10cl, 10c2, 10c3, 10c4, which may be stored on storage devices 20, 22, 24, 26 (respectively) coupled to client electronic devices 28, 30, 32, 34 (respectively), may be executed by one or more processors (not shown) and one or more memory architectures (not shown) incorporated into client electronic devices 28, 30, 32, 34 (respectively). Examples of storage devices 20, 22, 24, 26 may include but are not limited to: hard disk drives; tape drives; optical drives; RAID devices; random access memories (RAM); read-only memories (ROM), and all forms of flash memory storage devices.

Examples of client electronic devices 28, 30, 32, 34 may include, but are not limited to, data-enabled, cellular telephone 28, laptop computer 30, personal digital assistant 32, personal computer 34, a notebook computer (not shown), a server computer (not shown), a gaming console (not shown), a smart television (not shown), and a dedicated network device (not shown). Client electronic devices 28, 30, 32, 34 may each execute an operating system, examples of which may include but are not limited to Microsoft Windows™, Android™, WebOS™, iOS™, Redhat Linux™, or a custom operating system.

Additionally/alternatively, virtual desktops and virtual machines may be utilized to access charitable funding process 10. As is known in the art, a virtual machine (VM) is a software-based emulation of a physical computer, allowing multiple operating systems to run simultaneously on a single physical machine. It functions as an isolated environment that mimics the hardware of a computer, enabling users to install and operate operating systems and applications as if they were on a dedicated physical device. Virtual machines are managed by a hypervisor, which allocates resources such as CPU, memory, and storage to each VM while ensuring they operate independently from one another. This technology is widely used for software testing, running legacy applications, server consolidation, and creating secure environments. By abstracting hardware, virtual machines provide flexibility, scalability, and cost efficiency in both development and production settings. As is known in the art, a virtual desktop is a computing environment that allows users to access a desktop operating system and applications hosted on a remote server rather than a local device. Delivered through technologies like Virtual Desktop Infrastructure (VDI) or cloud services, a virtual desktop provides a consistent, customizable workspace accessible from various devices, including laptops, tablets, and smartphones. This setup enables centralized management of desktops, ensuring that updates, security, and backups are handled on the server side, enhancing IT efficiency and reducing maintenance costs. Virtual desktops are widely used for remote work, as they allow employees to securely access their work environment from anywhere, while businesses benefit from enhanced data security, scalability, and reduced hardware dependency.

Users 36, 38, 40, 42 may access charitable funding process 10 directly through network 14 or through secondary network 18. Further, charitable funding process 10 may be connected to network 14 through secondary network 18, as illustrated with link line 44.

The various client electronic devices (e.g., client electronic devices 28, 30, 32, 34) may be directly or indirectly coupled to network 14 (or network 18). For example, data-enabled, cellular telephone 28 and laptop computer 30 are shown wirelessly coupled to network 14 via wireless communication channels 46, 48 (respectively) established between data-enabled, cellular telephone 28, laptop computer 30 (respectively) and cellular network/bridge 50, which is shown directly coupled to network 14. Further, personal digital assistant 32 is shown wirelessly coupled to network 14 via wireless communication channel 52 established between personal digital assistant 32 and wireless access point (i.e., WAP) 54, which is shown directly coupled to network 14. Additionally, personal computer 34 is shown directly coupled to network 18 via a hardwired network connection.

WAP 54 may be, for example, an IEEE 802.11a, 802.11b, 802.11 g, 802.11n, Wi-Fi, and/or Bluetooth device that is capable of establishing wireless communication channel 52 between personal digital assistant 32 and WAP 54. As is known in the art, IEEE 802.11x specifications may use Ethernet protocol and carrier sense multiple access with collision avoidance (i.e., CSMA/CA) for path sharing. The various 802.11x specifications may use phase-shift keying (i.e., PSK) modulation or complementary code keying (i.e., CCK) modulation, for example. As is known in the art, Bluetooth is a telecommunications industry specification that allows e.g., mobile phones, computers, and personal digital assistants to be interconnected using a short-range wireless connection.

### Charitable Funding Process (Overview):

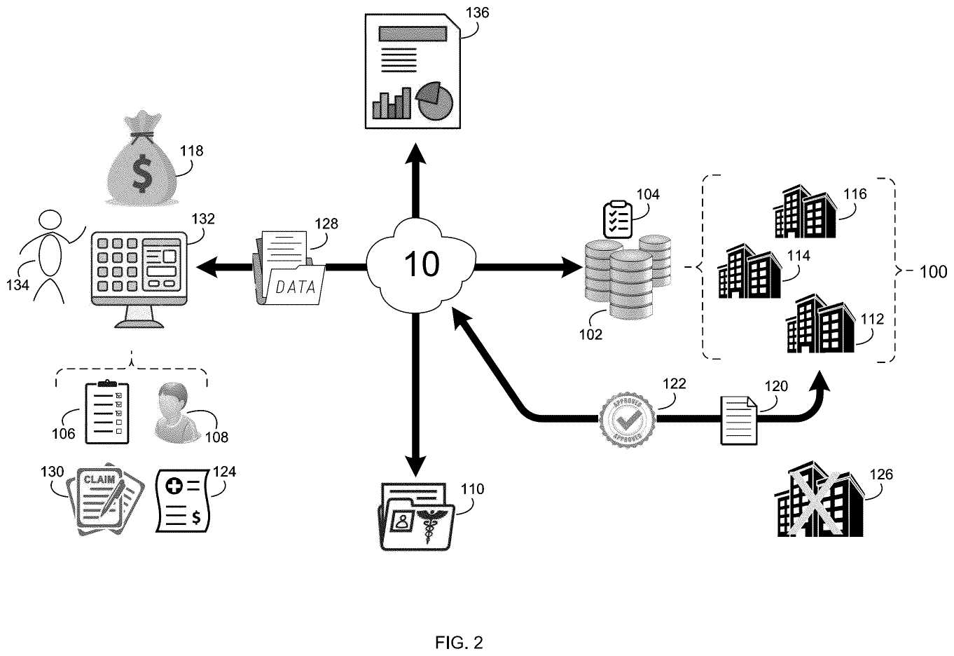

As discussed above and referring also to FIG. 2, charitable funding process 10 may be configured to match people in need of assistance with paying their medical expenses with charitable funds that offer such assistance. For example, assume that there are a plurality of charitable organizations (e.g., charitable organizations 100) that provide funding to those in need. Examples of these charitable organizations (e.g., charitable organizations 100) may include but are not limited to one or more of: a charity; a non-profit organization; and a philanthropic entity of a for-profit entity

When it comes to organizations that help fund medical expenses for people in need, a charity is a group established specifically for public benefit, such as providing financial support to cover medical costs. Charities often rely on donations, grants, and volunteer efforts to directly assist individuals or communities in need. They are typically tax-exempt and focus on directly addressing the financial challenges faced by those requiring medical care.

A non-profit organization (NPO) also works to fulfill a social, educational, or charitable mission, but its scope may extend beyond direct aid to include activities like advocacy, education, or community health programs. While charities are a subset of non-profits, not all non-profits qualify as charities. In the context of medical expenses, a non-profit might provide direct financial assistance but could also work on broader initiatives, such as raising awareness of healthcare resources or influencing policies that improve access to care.

A philanthropic entity of a for-profit organization refers to the charitable arm or foundation of a corporation. These entities allocate a portion of the company's profits or other resources to support social causes, including healthcare. Unlike charities or non-profits, these entities are directly linked to for-profit businesses and often align their giving with the company's mission or brand values. For instance, a pharmaceutical company's foundation might provide free medications or sponsor medical procedures for underserved communities. While all three types of organizations share a common goal of helping those in need, their structures, funding sources, and areas of focus differ significantly.

The funding provided by the plurality of charitable organizations (e.g., charitable organizations 100) may include one or more of: drug-based funding; treatment-based funding; and disease-based funding. Drug-based funding focuses on providing financial assistance for purchasing medications, offering free or subsidized access to essential drugs, or partnering with pharmaceutical companies to distribute medications to individuals who cannot afford them. This type of funding is particularly valuable for chronic conditions or rare diseases that require costly, long-term prescriptions. Treatment-based funding, on the other hand, supports broader medical interventions beyond medications, such as surgeries, hospital stays, rehabilitation, or specialized therapies. It ensures that patients can access the comprehensive care they need without financial barriers. Disease-based funding centers its support on specific illnesses or conditions, such as cancer, diabetes, or rare diseases.

Charitable organizations (e.g., charitable organizations 100) using this approach may also focus their resources on improving outcomes for affected individuals by funding research, patient education, early detection programs, and financial aid for related treatments or medications. Together, these funding models may enable charities to address the multifaceted needs of patients while maximizing the impact of their resources.

The one or more charitable organizations (e.g., charitable organizations 100) may be defined within a charitable organization database (e.g., charitable organization database 102). A charitable organization database (e.g., charitable organization database 102) may be a centralized collection of information about charities that serve various causes, including medical assistance, disaster relief, education, and more. These databases are designed to provide detailed profiles of charitable organizations (e.g., charitable organizations 100), such as their mission, areas of operation, programs, financial data, and contact information. They are often used by donors, grantmakers, researchers, and individuals seeking support to identify credible and effective charities. In the context of medical assistance, a charitable organization database (e.g., charitable organization database 102) may help users find organizations that fund specific medical treatments, provide grants for healthcare costs, or offer other forms of support. These databases can also include reviews, ratings, or impact metrics to help users evaluate the transparency and effectiveness of listed organizations. Accessible through online platforms, such databases may streamline the process of connecting those in need with resources and donors with trusted charities.

### Concept 1—The Platform 1

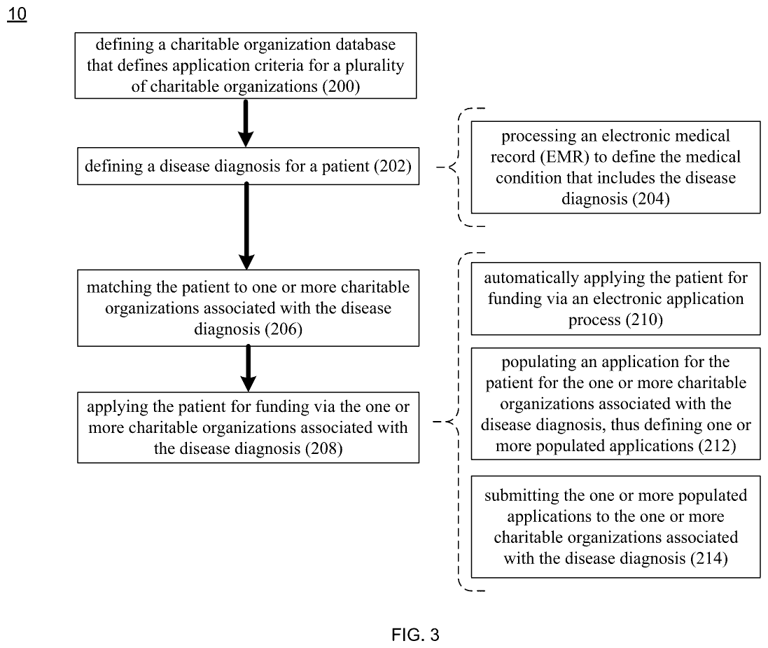

Referring also to FIG. 3, charitable funding process 10 may define 200 a charitable organization database (e.g., charitable organization database 102) that defines application criteria (e.g., application criteria 104) for a plurality of charitable organizations (e.g., charitable organizations 100). Examples of such application criteria (e.g., application criteria 104) may include but is not limited to disease criteria (e.g., disease type), income criteria (e.g., income limits), location/region criteria (e.g., state, country, geographic region), funding limit criteria (e.g., patient funding limits), expense type criteria (e.g., what funding may be sought for), patient age criteria (e.g., maximum/minimum age restrictions), as well as other various criteria.

Charitable funding process 10 may define 202 a disease diagnosis (e.g., disease diagnosis 106) for a patient (e.g., patient 108). A disease diagnosis is the process of identifying a specific illness or condition affecting a patient (e.g., patient 108) based on their symptoms, medical history, physical examination, and diagnostic tests. It involves a systematic evaluation by healthcare professionals to determine the underlying cause of a patient's health concerns. This process often begins with a detailed discussion about the patient's symptoms and lifestyle, followed by a physical assessment to look for clinical signs of illness. Diagnostic tools such as blood tests, imaging scans, biopsies, or genetic testing may be used to confirm the suspected condition. A precise diagnosis is critical for developing an effective treatment plan, managing symptoms, and improving patient outcomes. Additionally, a timely and accurate diagnosis can help prevent complications and guide decisions about preventive measures or long-term care strategies.

When defining 202 disease diagnosis (e.g., disease diagnosis 106) for a patient (e.g., patient 108), charitable funding process 10 may process 204 an electronic medical record (EMR) (e.g., electronic medical record 110) to define the medical condition that includes the disease diagnosis (e.g., disease diagnosis 106).

An Electronic Medical Record (EMR) (e.g., electronic medical record 110) is a digital version of a patient's medical history maintained by a healthcare provider. It includes comprehensive information such as demographics, medical history, medications, allergies, immunization records, lab test results, imaging studies, and treatment plans. EMRs play a crucial role in disease diagnosis (e.g., disease diagnosis 106) by providing quick and centralized access to a patient's health data, enabling more informed and accurate clinical decisions. For example, a doctor can review previous test results, track changes in a patient's health over time, and identify patterns or risk factors that could lead to a diagnosis (e.g., disease diagnosis 106). EMRs also facilitate collaboration among specialists by allowing them to share critical information, ensuring that all involved providers have a complete understanding of the patient's condition. Furthermore, integrated diagnostic tools within EMR systems can assist clinicians by offering decision support, flagging abnormalities, or suggesting potential diagnoses based on entered data. This efficiency and accessibility make EMRs an essential tool in modern healthcare, improving the accuracy and timeliness of disease diagnosis (e.g., disease diagnosis 106) and subsequent treatment planning.

Charitable funding process 10 may match 206 the patient (e.g., patient 108) to one or more charitable organizations (e.g., charitable organizations 100) associated with the disease diagnosis (e.g., disease diagnosis 106). As discussed above, the charitable organization database (e.g., charitable organization database 102) may define application criteria (e.g., application criteria 104) for a plurality of charitable organizations (e.g., charitable organizations 100), wherein each of these charitable organizations (e.g., charitable organizations 100) may be associated with a specific disease diagnosis or type of disease. For example, a first charitable organization (e.g., charitable organization 112) defined within charitable organization database (e.g., charitable organization database 102) may be associated with e.g., pediatric cancers, a second charitable organization (e.g., charitable organization 114) defined within charitable organization database (e.g., charitable organization database 102) may be associated with e.g., diabetes, and a third charitable organization (e.g., charitable organization 116) defined within charitable organization database (e.g., charitable organization database 102) may be associated with e.g., heart disease.

Accordingly, assume for this example that the patient (e.g., patient 108) is a four year old boy that was diagnosed with leukemia (e.g., disease diagnosis 106). Accordingly, charitable funding process 10 may match 206 the patient (e.g., patient 108) to the first charitable organization (e.g., charitable organization 112) defined within charitable organization database (e.g., charitable organization database 102), as it is associated with pediatric cancers.

Further, assume that the parents of the patient (e.g., patient 108) are in need of assistance concerning the medical expenses associated with the treatment of their son. Medical expenses encompass a wide range of costs incurred during the diagnosis, treatment, and management of health conditions. Surgical expenses include fees for surgical procedures, operating room charges, anesthesia, and pre- and post-operative care. These costs can vary widely depending on the complexity of the surgery and the facility. Physician expenses refer to fees for consultations, office visits, specialist care, and ongoing medical management provided by doctors or other healthcare professionals. Medication expenses cover the costs of prescription drugs, over-the-counter medications, and specialized treatments such as biologics or chemotherapy drugs, which can be significant for chronic or complex conditions. Hospital expenses include room and board during inpatient stays, emergency room visits, and ancillary services such as nursing care and facility fees. Lab expenses arise from diagnostic tests such as blood work, biopsies, or imaging scans like X-rays and MRIs, which are essential for diagnosing and monitoring diseases. Therapy expenses pertain to physical, occupational, or speech therapy services needed for rehabilitation or management of chronic conditions. Beyond direct medical care, additional expenses can arise, such as transportation expenses, which cover the cost of traveling to and from medical appointments or facilities, particularly for those requiring long-distance or frequent travel. Housing expenses may be incurred when patients or their families need temporary accommodations near a treatment facility, such as during extended hospital stays or clinical trials. These diverse categories of medical expenses highlight the multifaceted financial burdens that patients and families may face during healthcare journeys.

Charitable funding process 10 may apply 208 the patient (e.g., patient 108) for funding (e.g., funding 118) via the one or more charitable organizations (e.g., charitable organizations 100) associated with the disease diagnosis (e.g., disease diagnosis 106). For example, charitable funding process 10 may apply 208 the parents of the patient (e.g., patient 108) for funding (e.g., funding 118) via the first charitable organization (e.g., charitable organization 112) defined within charitable organization database (e.g., charitable organization database 102), as charitable organization 112 is associated with pediatric cancers.

When applying 208 the patient (e.g., patient 108) for funding (e.g., funding 118) via the one or more charitable organizations (e.g., charitable organizations 100), charitable funding process 10 may automatically apply 210 the patient (e.g., patient 108) for funding (e.g., funding 118) via an electronic application process.

An electronic application process is an online method for submitting requests to a charitable organization for financial assistance with medical expenses. This process allows applicants to complete and submit required forms digitally through a website or application portal, making it more accessible and efficient than traditional paper-based applications. Applicants typically provide personal information, medical details, proof of income or financial need, and documentation such as medical bills, insurance statements, or physician recommendations.

The electronic application process often includes features such as guided instructions, pre-filled forms for returning users, and the ability to upload supporting documents directly. Some systems also offer status tracking, enabling applicants to monitor the progress of their requests in real-time. By streamlining data collection and submission, this process reduces administrative overhead for both the applicant and the charitable organization, ensuring quicker review and decision-making. Additionally, the electronic format enhances accessibility for individuals who may live far from the charity's physical office or need urgent assistance.

Generally and when applying 208 the patient (e.g., patient 108) for funding (e.g., funding 118) via the one or more charitable organizations (e.g., charitable organizations 100) associated with the disease diagnosis (e.g., disease diagnosis 106), charitable funding process 10 may populate 212 an application for the patient (e.g., patient 108) for the one or more charitable organizations (e.g., charitable organizations 100) associated with the disease diagnosis (e.g., disease diagnosis 106), thus defining one or more populated applications (e.g., application(s) 120).

Further and when applying 208 the patient (e.g., patient 108) for funding (e.g., funding 118) via the one or more charitable organizations (e.g., charitable organizations 100) associated with the disease diagnosis (e.g., disease diagnosis 106), charitable funding process 10 may submit 214 the one or more populated applications (e.g., application(s) 120) to the one or more charitable organizations (e.g., charitable organizations 100) associated with the disease diagnosis (e.g., disease diagnosis 106).

Continuing with the above-stated example, charitable funding process 10 may populate 212 an application (e.g., application(s) 120) for patient 108 for charitable organization 112 associated with the disease diagnosis (e.g., disease diagnosis 106), wherein charitable funding process 10 may submit 214 the application (e.g., application(s) 120) to charitable organization 112, which is associated with the disease diagnosis (e.g., disease diagnosis 106).

Once charitable funding process 10 applies 208 the patient (e.g., patient 108) for funding (e.g., funding 118) via the one or more charitable organizations (e.g., charitable organizations 100) associated with the disease diagnosis (e.g., disease diagnosis 106), charitable funding process 10 may obtain 216 a funding approval (e.g., funding approval 122) from the one or more charitable organizations (e.g., charitable organizations 100).

Continuing with the above-stated example, if charitable funding process 10 obtains 216 such a funding approval (e.g., funding approval 122) from the one or more charitable organizations (e.g., charitable organizations 100), the parents of patient 108 may receive such funding (e.g., funding 118) and utilize the same to satisfy medical expenses (e.g., medical expenses 124) associated with the treatment of their son. As discussed above, examples of such medical expenses (e.g., medical expenses 124) may include but are not limited to: surgical expenses; physician expenses; medication expenses; hospital expenses; lab expenses; therapy expenses; transportation expenses; and housing expenses.

### Concept 2—The Platform 2

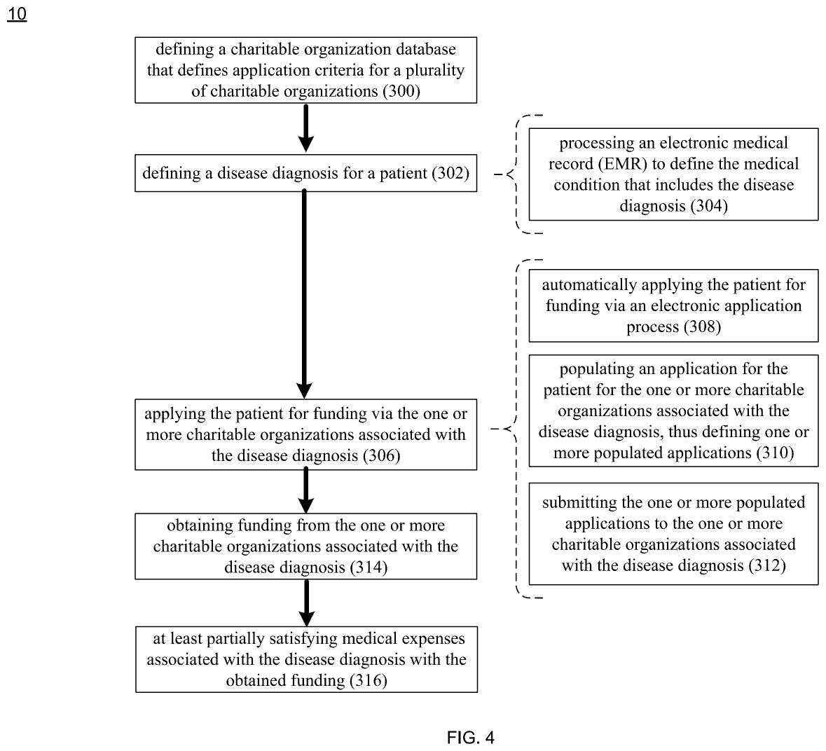

Referring also to FIG. 4 and as discussed above, charitable funding process 10 may define 300 a charitable organization database (e.g., charitable organization database 102) that defines application criteria (e.g., application criteria 104) for a plurality of charitable organizations (e.g., charitable organizations 100). Examples of such application criteria (e.g., application criteria 104) may include but is not limited to disease criteria (e.g., disease type), income criteria (e.g., income limits), location/region criteria (e.g., state, country, geographic region), funding limit criteria (e.g., patient funding limits), expense type criteria (e.g., what funding may be sought for), patient age criteria (e.g., maximum/minimum age restrictions), as well as other various criteria.

As discussed above, a charitable organization database (e.g., charitable organization database 102) may be a centralized collection of information about charities that serve various causes, including medical assistance, disaster relief, education, and more. These databases are designed to provide detailed profiles of charitable organizations (e.g., charitable organizations 100), and are often used by donors, grantmakers, researchers, and individuals seeking support to identify credible and effective charities. In the context of medical assistance, a charitable organization database (e.g., charitable organization database 102) may help users find organizations that fund specific medical treatments, provide grants for healthcare costs, or offer other forms of support.

Charitable funding process 10 may define 302 a disease diagnosis (e.g., disease diagnosis 106) for a patient (e.g., patient 108). As discussed above, a disease diagnosis is the process of identifying a specific illness or condition affecting a patient (e.g., patient 108) based on their symptoms, medical history, physical examination, and diagnostic tests, wherein it often involves a systematic evaluation by healthcare professionals to determine the underlying cause of a patient's health concerns.

When defining 302 a disease diagnosis (e.g., disease diagnosis 106) for a patient (e.g., patient 108), charitable funding process 10 may process 304 an electronic medical record (EMR) (e.g., electronic medical record 110) to define the medical condition that includes the disease diagnosis (e.g., disease diagnosis 106).

As discussed above, an Electronic Medical Record (EMR) (e.g., electronic medical record 110) is a digital version of a patient's medical history maintained by a healthcare provider, which includes comprehensive information such as demographics, medical history, medications, allergies, immunization records, lab test results, imaging studies, and treatment plans. EMRs play a crucial role in disease diagnosis (e.g., disease diagnosis 106) by providing quick and centralized access to a patient's health data, enabling more informed and accurate clinical decisions.

Charitable funding process 10 may apply 306 the patient (e.g., patient 108) for funding (e.g., funding 118) via one or more charitable organizations (e.g., charitable organizations 100) associated with the disease diagnosis (e.g., disease diagnosis 106). For example and as discussed above, charitable funding process 10 may apply 306 the parents of the patient (e.g., patient 108) for funding (e.g., funding 118) via the first charitable organization (e.g., charitable organization 112) defined within charitable organization database (e.g., charitable organization database 102), as charitable organization 112 is associated with pediatric cancers.

As discussed above and when applying 306 patient (e.g., patient 108) for funding (e.g., funding 118) via the one or more charitable organizations (e.g., charitable organizations 100), charitable funding process 10 may automatically apply 308 the patient (e.g., patient 108) for funding (e.g., funding 118) via an electronic application process. As discussed above, an electronic application process is an online method for submitting requests to a charitable organization for financial assistance with medical expenses (e.g., medical expenses 124). This process allows applicants to complete and submit required forms digitally through a website or application portal, making it more accessible and efficient than traditional paper-based applications.

As discussed above and when applying 306 the patient (e.g., patient 108) for funding (e.g., funding 118) via the one or more charitable organizations (e.g., charitable organizations 100) associated with the disease diagnosis (e.g., disease diagnosis 106), charitable funding process 10 may populate 310 an application for the patient (e.g., patient 108) for the one or more charitable organizations (e.g., charitable organizations 100) associated with the disease diagnosis (e.g., disease diagnosis 106), thus defining one or more populated applications (e.g., application(s) 120).

As discussed above and when applying 306 the patient (e.g., patient 108) for funding (e.g., funding 118) via the one or more charitable organizations (e.g., charitable organizations 100) associated with the disease diagnosis (e.g., disease diagnosis 106), charitable funding process 10 may submit 312 the one or more populated applications (e.g., application(s) 120) to the one or more charitable organizations (e.g., charitable organizations 100) associated with the disease diagnosis (e.g., disease diagnosis 106).

Continuing with the above-stated example, charitable funding process 10 may populate 310 an application (e.g., application(s) 120) for patient 108 for charitable organization 112 associated with the disease diagnosis (e.g., disease diagnosis 106), wherein charitable funding process 10 may submit 312 the application (e.g., application(s) 120) to charitable organization 112, which is associated with the disease diagnosis (e.g., disease diagnosis 106).

In response to applying 306 the patient (e.g., patient 108) for funding (e.g., funding 118), charitable funding process 10 may obtain 314 funding (e.g., funding 118) from the one or more charitable organizations (e.g., charitable organizations 100) associated with the disease diagnosis (e.g., disease diagnosis 106). And if such funding (e.g., funding 118) is obtained 314 from the one or more charitable organizations (e.g., charitable organizations 100), charitable funding process 10 may at least partially satisfy 316 medical expenses (e.g., medical expenses 124) associated with the disease diagnosis (e.g., disease diagnosis 106) with the obtained funding (e.g., funding 118)

Continuing with the above-stated example, if charitable funding process 10 obtains 314 such a funding approval (e.g., funding approval 122) from charitable organization 112, the parents of patient 108 may receive such funding (e.g., funding 118), wherein charitable funding process 10 may utilize such funding (e.g., funding 118) to at least partially satisfy 316 the medical expenses (e.g., medical expenses 124) associated with the disease diagnosis (e.g., disease diagnosis 106).

### Concept 3—Auto-Enrollment in a Reopened Fund

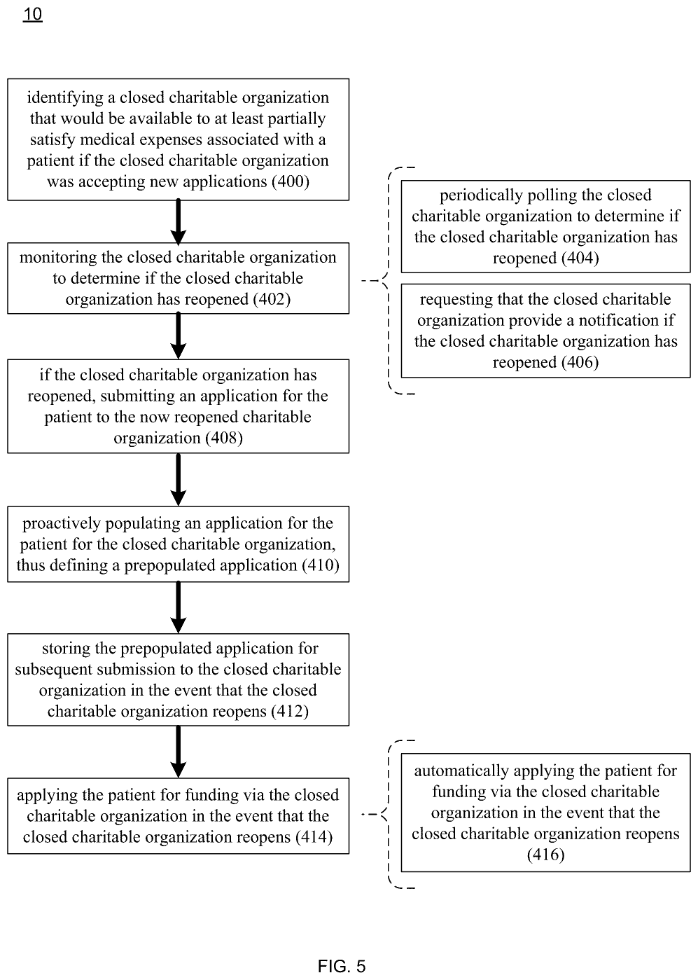

Referring also to FIG. 5, charitable funding process 10 may identify 400 a closed charitable organization (e.g., closed charitable organization 126) that would be available to at least partially satisfy medical expenses (e.g., medical expenses 124) associated with a patient (e.g., patient 108) if the closed charitable organization (e.g., closed charitable organization 126) was accepting new applications.

A closed charitable organization (e.g., closed charitable organization 126) refers to a charity that is no longer accepting applications for financial assistance, such as funding for medical expenses. This closure can be temporary or permanent and may occur for various reasons, including depletion of funds, restructuring of programs, or the conclusion of its mission or operational period. When a charitable organization closes its application process, it typically stops reviewing new requests but may continue to honor commitments made to previously approved applicants. For individuals seeking help, this can mean redirecting their efforts to other organizations or resources. Charities usually announce such closures through their websites, social media platforms, or public notices, often providing explanations or alternative options for those in need. This situation underscores the importance of staying informed about available resources and acting promptly when seeking financial assistance.

Continuing with the example in which the patient (e.g., patient 108) is a four year old boy that was diagnosed with leukemia (e.g., disease diagnosis 106) and the first charitable organization (e.g., charitable organization 112) is associated with pediatric cancers, assume that the closed charitable organization (e.g., closed charitable organization 126) is the first charitable organization (e.g., charitable organization 112).

Charitable funding process 10 may monitor 402 the closed charitable organization (e.g., closed charitable organization 126) to determine if the closed charitable organization (e.g., closed charitable organization 126) has reopened.

For example and when monitoring 402 the closed charitable organization (e.g., closed charitable organization 126) to determine if the closed charitable organization (e.g., closed charitable organization 126) has reopened, charitable funding process 10 may periodically poll 404 the closed charitable organization (e.g., closed charitable organization 126) to determine if the closed charitable organization (e.g., closed charitable organization 126) has reopened.

To periodically poll a closed charitable organization (e.g., closed charitable organization 126) means to regularly contact or check in with the organization to determine if they have reopened their application process for financial assistance. This can involve automated visits to their website or sending follow-up emails to inquire about the status of their funding availability. Polling is often done when an organization has temporarily closed its application process due to limited resources or operational constraints, but there is a possibility that they may reopen in the future. This approach ensures that individuals remain updated on any changes in the organization's policies or funding status, allowing them to act promptly when applications are accepted again.

Further and when monitoring 402 closed charitable organization (e.g., closed charitable organization 126) to determine if the closed charitable organization (e.g., closed charitable organization 126) has reopened, charitable funding process 10 may request 406 that the closed charitable organization (e.g., closed charitable organization 126) provide a notification if the closed charitable organization (e.g., closed charitable organization 126) has reopened.

For example, certain closed charitable organizations (e.g., closed charitable organization 126) may enable a user to e.g., be placed on a waiting list so that they are automatically notified in the event that the closed charitable organization (e.g., closed charitable organization 126) has reopened.

If the closed charitable organization (e.g., closed charitable organization 126) has reopened, charitable funding process 10 may submit 408 an application (e.g., application(s) 120) for the patient (e.g., patient 108) to the now reopened charitable organization. For example, charitable funding process 10 may submit 408 the application (e.g., application(s) 120) for the patient (e.g., patient 108) to charitable organization 112 (i.e., the now reopened charitable organization), which is associated with the disease diagnosis (e.g., disease diagnosis 106).

If the closed charitable organization (e.g., closed charitable organization 126) has not yet reopened, charitable funding process 10 may proactively populate 410 an application for the patient (e.g., patient 108) for the closed charitable organization (e.g., closed charitable organization 126), thus defining a prepopulated application (e.g., application(s) 120). Once populated 410, charitable funding process 10 may store 412 the prepopulated application (e.g., application(s) 120) for subsequent submission to the closed charitable organization (e.g., closed charitable organization 126) in the event that the closed charitable organization (e.g., closed charitable organization 126) reopens.

Once stored 412, charitable funding process 10 may apply 414 the patient (e.g., patient 108) for funding (e.g., funding 118) via the closed charitable organization (e.g., closed charitable organization 126) in the event that the closed charitable organization (e.g., closed charitable organization 126) reopens. When applying 414 the patient (e.g., patient 108) for funding (e.g., funding 118) via the closed charitable organization (e.g., closed charitable organization 126) in the event that the closed charitable organization (e.g., closed charitable organization 126) reopens, charitable funding process 10 may automatically apply 416 the patient (e.g., patient 108) for funding (e.g., funding 118) via the closed charitable organization (e.g., closed charitable organization 126) in the event that the closed charitable organization (e.g., closed charitable organization 126) reopens.

Such automatic application submission for funding may be effectuated via an electronic application process. As discussed above, an electronic application process is an online method for submitting requests to a charitable organization for financial assistance with medical expenses. This process allows applicants to complete and submit required forms digitally through a website or application portal, making it more accessible and efficient than traditional paper-based applications. Applicants typically provide personal information, medical details, proof of income or financial need, and documentation such as medical bills, insurance statements, or physician recommendations.

### Concept 4—Reactively Processing Outstanding Bills

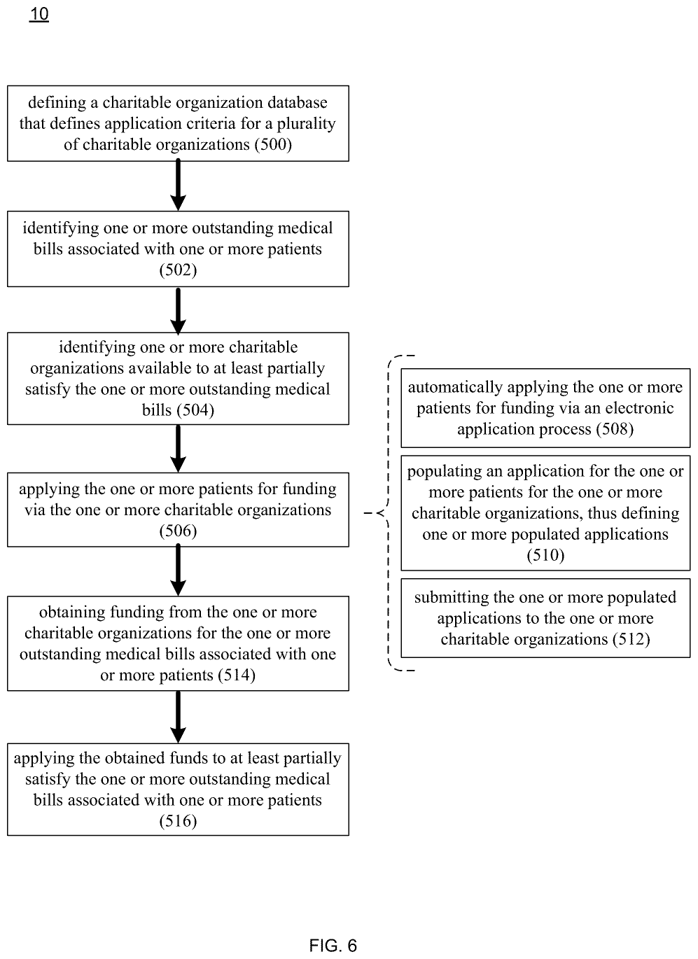

Referring also to FIG. 6, charitable funding process 10 may define 500 a charitable organization database (e.g., charitable organization database 102) that defines application criteria (e.g., application criteria 104) for a plurality of charitable organizations (e.g., charitable organizations 100). Examples of such application criteria (e.g., application criteria 104) may include but is not limited to disease criteria (e.g., disease type), income criteria (e.g., income limits), location/region criteria (e.g., state, country, geographic region), funding limit criteria (e.g., patient funding limits), expense type criteria (e.g., what funding may be sought for), patient age criteria (e.g., maximum/minimum age restrictions), as well as other various criteria.

As discussed above, a charitable organization database (e.g., charitable organization database 102) may be a centralized collection of information about charities that serve various causes, including medical assistance, disaster relief, education, and more. These databases are designed to provide detailed profiles of charitable organizations (e.g., charitable organizations 100), and are often used by donors, grantmakers, researchers, and individuals seeking support to identify credible and effective charities. In the context of medical assistance, a charitable organization database (e.g., charitable organization database 102) may help users find organizations that fund specific medical treatments, provide grants for healthcare costs, or offer other forms of support.

Charitable funding process 10 may identify 502 one or more outstanding medical bills (e.g., medical expenses 124) associated with one or more patients (e.g., patient 108). For example, charitable funding process 10 may enable the patient (e.g., patient 108) to identify 502 the one or more outstanding medical bills (e.g., medical expenses 124) by e.g., scanning them/submitting them/uploading them/defining them so that these outstanding medical bills may be processed by charitable funding process 10.

Once these outstanding medical bills (e.g., medical expenses 124) are identified 502, charitable funding process 10 may identify 504 one or more charitable organizations (e.g., charitable organizations 100) available to at least partially satisfy the one or more outstanding medical bills (e.g., medical expenses 124).

As discussed above, the charitable organization database (e.g., charitable organization database 102) may define application criteria (e.g., application criteria 104) for a plurality of charitable organizations (e.g., charitable organizations 100), wherein each of these charitable organizations (e.g., charitable organizations 100) may be associated with a specific disease diagnosis or type of disease.

Continuing with the above-stated example in which the patient (e.g., patient 108) is a four year old boy that was diagnosed with leukemia (e.g., disease diagnosis 106), charitable funding process 10 may identify 504 the first charitable organization (e.g., charitable organization 112) defined within charitable organization database (e.g., charitable organization database 102) as a charitable organization that is available to at least partially satisfy the one or more outstanding medical bills (e.g., medical expenses 124), since charitable organization 112 is associated with pediatric cancers.

As discussed above, charitable funding process 10 may apply 506 the one or more patients (e.g., patient 108) for funding (e.g., funding 118) via the one or more charitable organizations (e.g., charitable organizations 100). When applying 506 the one or more patients (e.g., patient 108) for funding (e.g., funding 118) via the one or more charitable organizations (e.g., charitable organizations 100), charitable funding process 10 may automatically apply 508 the one or more patients (e.g., patient 108) for funding (e.g., funding 118) via an electronic application process.

As discussed above, such an automatic application for funding may be effectuated via an electronic application process. As discussed above, an electronic application process is an online method for submitting requests to a charitable organization for financial assistance with medical expenses. This process allows applicants to complete and submit required forms digitally through a website or application portal, making it more accessible and efficient than traditional paper-based applications. Applicants typically provide personal information, medical details, proof of income or financial need, and documentation such as medical bills, insurance statements, or physician recommendations

When applying 506 the one or more patients (e.g., patient 108) for funding (e.g., funding 118) via the one or more charitable organizations (e.g., charitable organizations 100), charitable funding process 10 may populate 510 an application for the one or more patients (e.g., patient 108) for the one or more charitable organizations (e.g., charitable organizations 100), thus defining one or more populated applications (e.g., application(s) 120).

Further and when applying 506 the one or more patients (e.g., patient 108) for funding (e.g., funding 118) via the one or more charitable organizations (e.g., charitable organizations 100), charitable funding process 10 may submit 512 the one or more populated applications (e.g., application(s) 120) to the one or more charitable organizations (e.g., charitable organizations 100).

In response to applying 506 the patient (e.g., patient 108) for funding (e.g., funding 118), charitable funding process 10 may obtain 514 funding (e.g., funding 118) from the one or more charitable organizations (e.g., charitable organizations 100) for the one or more outstanding medical bills (e.g., medical expenses 124) associated with one or more patients (e.g., patient 108).

And if such funding (e.g., funding 118) is obtained 514 from the one or more charitable organizations (e.g., charitable organizations 100), charitable funding process 10 may apply 516 the obtained funds (e.g., funding 118) to at least partially satisfy the one or more outstanding medical bills (e.g., medical expenses 124) associated with one or more patients (e.g., patient 108).

### Concept 5—Status Change Notification

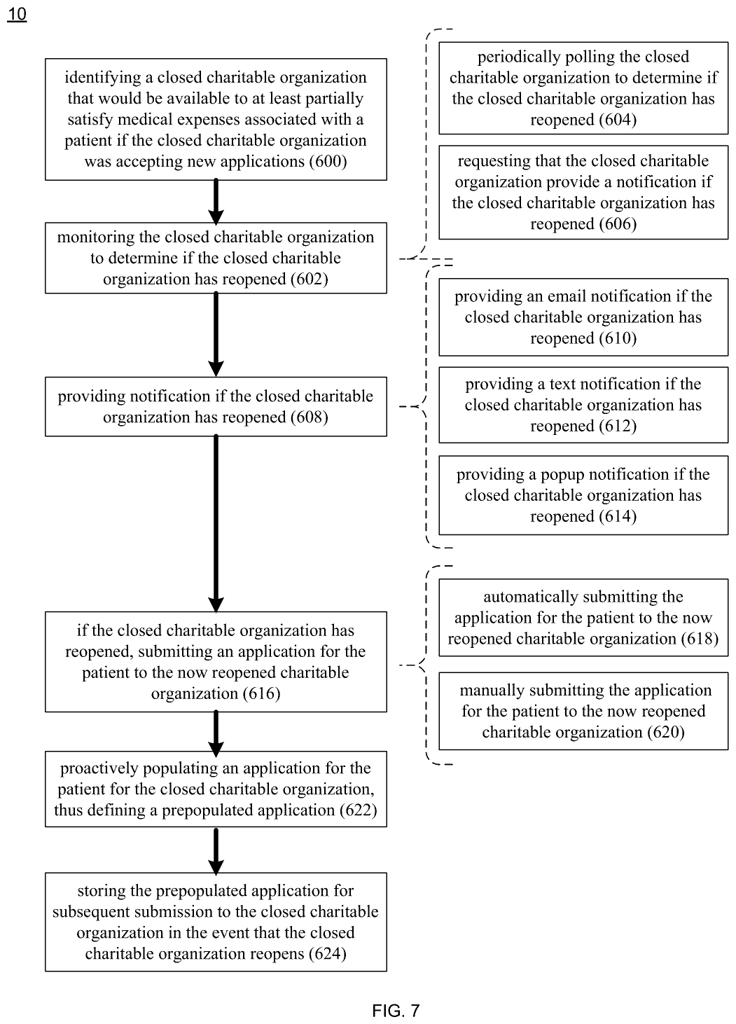

Referring also to FIG. 7, charitable funding process 10 may identify 600 a closed charitable organization (e.g., closed charitable organization 126) that would be available to at least partially satisfy medical expenses (e.g., medical expenses 124) associated with a patient (e.g., patient 108) if the closed charitable organization (e.g., closed charitable organization 126) was accepting new applications.

A closed charitable organization (e.g., closed charitable organization 126) refers to a charity that is no longer accepting applications for financial assistance, such as funding for medical expenses. This closure can be temporary or permanent and may occur for various reasons, including depletion of funds, restructuring of programs, or the conclusion of its mission or operational period. When a charitable organization closes its application process, it typically stops reviewing new requests but may continue to honor commitments made to previously approved applicants. For individuals seeking help, this can mean redirecting their efforts to other organizations or resources. Charities usually announce such closures through their websites, social media platforms, or public notices, often providing explanations or alternative options for those in need. This situation underscores the importance of staying informed about available resources and acting promptly when seeking financial assistance.

Continuing with the example in which the patient (e.g., patient 108) is a four year old boy that was diagnosed with leukemia (e.g., disease diagnosis 106) and the first charitable organization (e.g., charitable organization 112) is associated with pediatric cancers, assume that the closed charitable organization (e.g., closed charitable organization 126) is the first charitable organization (e.g., charitable organization 112).

Charitable funding process 10 may monitor 602 the closed charitable organization (e.g., closed charitable organization 126) to determine if the closed charitable organization (e.g., closed charitable organization 126) has reopened.

For example and when monitoring 602 the closed charitable organization (e.g., closed charitable organization 126) to determine if the closed charitable organization (e.g., closed charitable organization 126) has reopened, charitable funding process 10 may periodically poll 604 the closed charitable organization (e.g., closed charitable organization 126) to determine if the closed charitable organization (e.g., closed charitable organization 126) has reopened.

As discussed above, to periodically poll a closed charitable organization (e.g., closed charitable organization 126) means to regularly contact or check in with the organization to determine if they have reopened their application process for financial assistance. This can involve automated visits to their website or sending follow-up emails to inquire about the status of their funding availability. Polling is often done when an organization has temporarily closed its application process due to limited resources or operational constraints, but there is a possibility that they may reopen in the future. This approach ensures that individuals remain updated on any changes in the organization's policies or funding status, allowing them to act promptly when applications are accepted again.

Alternatively and when monitoring 602 the closed charitable organization (e.g., closed charitable organization 126) to determine if the closed charitable organization (e.g., closed charitable organization 126) has reopened, charitable funding process 10 may request 606 that the closed charitable organization (e.g., closed charitable organization 126) provide a notification if the closed charitable organization (e.g., closed charitable organization 126) has reopened.

For example, certain closed charitable organizations (e.g., closed charitable organization 126) may enable a user to e.g., be placed on a waiting list so that they are automatically notified in the event that the closed charitable organization (e.g., closed charitable organization 126) has reopened.

Charitable funding process 10 may provide 608 notification if the closed charitable organization (e.g., closed charitable organization 126) has reopened.

For example and when providing 608 notification if the closed charitable organization (e.g., closed charitable organization 126) has reopened, charitable funding process 10 may: provide 610 an email notification if the closed charitable organization (e.g., closed charitable organization 126) has reopened.

An email notification in this context is a digital alert sent by a charitable organization to inform individuals that it has reopened its application process for funding medical expenses. This system allows people who have previously expressed interest or signed up for updates to receive timely information about changes in the organization's status. Email notifications typically include key details, such as the date applications will be accepted, eligibility criteria, required documentation, and submission deadlines. For individuals seeking assistance, signing up for email notifications is an effective way to stay informed without the need to repeatedly check the organization's website or contact them directly. Many charitable organizations offer an opt-in feature on their websites where users can provide their email addresses to subscribe to updates. When the application process reopens, the notification ensures that potential applicants can act quickly to prepare and submit their applications, especially if funding is limited and distributed on a first-come, first-served basis. This streamlined communication method is a convenient way for organizations to reach a broad audience while ensuring applicants stay informed.

Further and when providing 608 notification if the closed charitable organization (e.g., closed charitable organization 126) has reopened, charitable funding process 10 may: provide 612 a text notification if the closed charitable organization (e.g., closed charitable organization 126) has reopened.

A text notification in this context is a short message sent via SMS to inform individuals that a charitable organization, previously closed to applications, is now accepting new applications for financial assistance with medical expenses. Text notifications are a fast and direct way to reach individuals who have opted in to receive updates, ensuring they are promptly informed about changes to the organization's application status. These notifications typically include essential details, such as the reopening date, a link to the application portal, key eligibility requirements, and any important deadlines. Because text messages are easily accessible on mobile devices, they provide a convenient and immediate method of communication for both the organization and the recipients. For individuals seeking assistance, subscribing to text notifications helps them stay informed without needing to frequently check websites or make calls. It also ensures that they can act quickly to submit their applications, especially if funding is limited or distributed on a first-come, first-served basis. This approach enhances accessibility and communication, particularly for those who rely on mobile devices as their primary means of staying connected.

Additionally and when providing 608 notification if the closed charitable organization (e.g., closed charitable organization 126) has reopened, charitable funding process 10 may; and provide 614 a popup notification if the closed charitable organization (e.g., closed charitable organization 126) has reopened.

A popup notification in this context is a real-time alert displayed on a user's device, such as a computer, smartphone, or tablet, informing them that a charitable organization previously closed to applications is now accepting new ones for funding medical expenses. These notifications typically appear as small windows or banners on the screen, even when the user is not actively engaging with the organization's website or app. They are often part of a subscription service linked to a web browser, mobile application, or desktop application, which users opt into to receive timely updates. Popup notifications provide concise and actionable information, such as the application reopening date, a direct link to the application form, and reminders about eligibility requirements or deadlines. They are particularly effective for reaching users quickly, as the notification appears immediately, reducing the risk of missing important updates. For individuals seeking financial assistance, enabling popup notifications ensures they receive prompt alerts without needing to actively monitor the organization's communication channels, making it easier to respond promptly and submit applications.

If the closed charitable organization (e.g., closed charitable organization 126) has reopened, charitable funding process 10 may submit 616 an application for the patient (e.g., patient 108) to the now reopened charitable organization. For example, charitable funding process 10 may submit 616 the application (e.g., application(s) 120) for the patient (e.g., patient 108) to charitable organization 112 (i.e., the now reopened charitable organization), which is associated with the disease diagnosis (e.g., disease diagnosis 106).

When submitting 616 an application for the patient (e.g., patient 108) to the now reopened charitable organization, charitable funding process 10 may automatically submit 618 the application (e.g., application(s) 120) for the patient (e.g., patient 108) to the now reopened charitable organization.

As discussed above, such an automatic application submission for funding may be effectuated via an electronic application process. As discussed above, an electronic application process is an online method for submitting requests to a charitable organization for financial assistance with medical expenses. This process allows applicants to complete and submit required forms digitally through a website or application portal, making it more accessible and efficient than traditional paper-based applications. Applicants typically provide personal information, medical details, proof of income or financial need, and documentation such as medical bills, insurance statements, or physician recommendations

When submitting 616 an application for the patient (e.g., patient 108) to the now reopened charitable organization, charitable funding process 10 may manually submit 620 the application (e.g., application(s) 120) for the patient (e.g., patient 108) to the now reopened charitable organization.

For example, such a manual application submission for funding may be effectuated via the assistance of a data entry person that may manually upload the application (e.g., application(s) 120) to (in this example) charitable organization 112 (i.e., the now reopened charitable organization).

As discussed above and in order to effectuate a timely submission of an application in the event that the closed charitable organization (e.g., closed charitable organization 126) was accepting new applications, charitable funding process 10 may proactively populate 622 an application (e.g., application(s) 120) for the patient (e.g., patient 108) for the closed charitable organization (e.g., closed charitable organization 126), thus defining a prepopulated application (e.g., application(s) 120); and store 624 the prepopulated application (e.g., application(s) 120) for subsequent submission to the closed charitable organization (e.g., closed charitable organization 126) in the event that the closed charitable organization (e.g., closed charitable organization 126) reopens.

### Concept 6—Payment Status User Interface

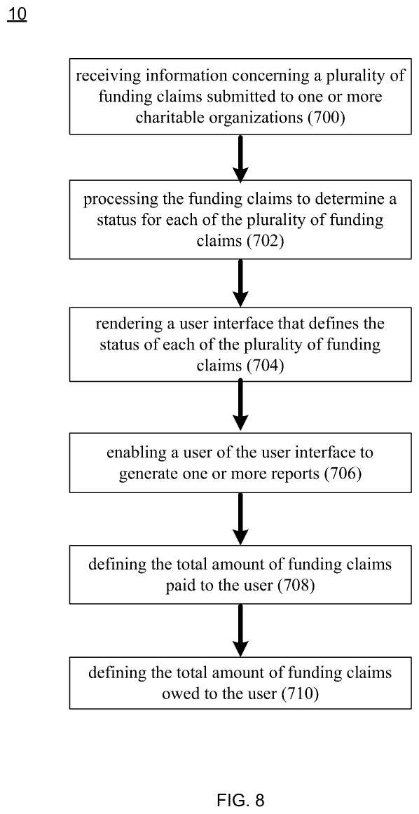

Referring also to FIG. 8, charitable funding process 10 may receive 700 information (e.g., information 128) concerning a plurality of funding claims (e.g., funding claims 130) submitted to one or more charitable organizations (e.g., charitable organizations 100).

Continuing with the above-stated example in which the patient (e.g., patient 108) is a four year old boy that was diagnosed with leukemia (e.g., disease diagnosis 106), assume that charitable funding process 10 receives 700 information (e.g., information 128) concerning a plurality of funding claims (e.g., funding claims 130) submitted to one or more charitable organizations (e.g., charitable organizations 100). Further, assume that this plurality of funding claims (e.g., funding claims 130) concern claims made by the parents of the patient (e.g., patient 108) to the first charitable organization (e.g., charitable organization 112) defined within charitable organization database (e.g., charitable organization database 102), as it is associated with pediatric cancers.

Charitable funding process 10 may process 702 the funding claims (e.g., funding claims 130) to determine a status for each of the plurality of funding claims (e.g., funding claims 130). The status of each of the plurality of funding claims may define one or more of: a portion of the plurality of funding claims that have been submitted but not yet approved; a portion of the plurality of funding claims that have been approved but not yet paid; and a portion of the plurality of funding claims that have been paid.

In the context of funding claims submitted to a charitable organization for medical expenses, there are distinct stages that claims can go through. A portion of the plurality of funding claims that have been submitted but not yet approved refers to claims that applicants have submitted and are currently under review or processing by the organization. These claims are in the pending stage, where eligibility and compliance with the organization's requirements are still being evaluated, and no decision has been made regarding approval. A portion of the plurality of funding claims that have been approved but not yet paid includes claims that have successfully passed the review process and been granted approval for financial assistance but for which the actual disbursement of funds has not yet occurred. These claims are awaiting payment, which may be delayed due to administrative processing or other factors. Lastly, a portion of the plurality of funding claims that have been paid represents claims that have been both approved and fully funded. These are completed transactions where the promised financial assistance has been disbursed to the intended recipient, whether it is the applicant, a healthcare provider, or a medical facility.

Charitable funding process 10 may render 704 a user interface (e.g., user interface 132) that defines the status of each of the plurality of funding claims (e.g., funding claims 130). An example of the user interface (e.g., user interface 132) may include but is not limited to a graphical user interface.

A graphical user interface (GUI) designed to help users track the status of their funding claims could include several intuitive features. One example is a progress bar with status indicators, where each stage of the process—such as “Submitted,” “Under Review,” “Approved,” and “Paid”—is represented by a segment. As the claim advances, the corresponding segment is highlighted, allowing users to easily see where their claim stands. Another option is a dashboard with summary cards that display key metrics at a glance, such as the number of claims in each stage. Each claim could be represented by a card that includes details like the claim ID, submission date, and current status, with clickable options for more information. A table view with filtering and sorting options provides another method, listing all claims with columns for claim ID, submission date, status, and amount, enabling users to filter by status or sort by date for easier navigation. An interactive timeline could visually map out the journey of a claim through various stages, with dates and details accessible by hovering or clicking on specific points. Notifications and alerts are another essential feature; a notifications panel could provide real-time updates on changes to claim statuses, color-coded for urgency, such as green for progress or yellow for required user action. Additionally, a claim tracking widget with color coding might group claims by status, using distinct colors for categories like “Pending,” “Approved,” and “Paid,” while allowing users to click each group to view individual claims. For mobile users, a mobile-friendly interface with large icons representing statuses—such as a magnifying glass for “Under Review” or a dollar sign for “Paid”—and quick action buttons for tasks like submitting missing documents ensures accessibility and ease of use. These features may collectively enhance transparency, efficiency, and user experience in tracking funding claims.

Charitable funding process 10 may enable 706 a user (e.g., user 134) of the user interface (e.g., user interface 132) to generate one or more reports (e.g., reports 136), wherein these one or more reports (e.g., reports 136) may be based upon a defined period of time. Additionally/alternatively, charitable funding process 10 may define 708 the total amount of funding claims (e.g., funding claims 130) paid to the user (e.g., user 132), as well as define 710 the total amount of funding claims (e.g., funding claims 130) owed to the user (e.g., user 132).

### General:

As will be appreciated by one skilled in the art, the present disclosure may be embodied as a method, a system, or a computer program product. Accordingly, the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present disclosure may take the form of a computer program product on a computer-usable storage medium having computer-usable program code embodied in the medium.

Any suitable computer usable or computer readable medium may be utilized. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. The computer-usable or computer-readable medium may also be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, RF, etc.

Computer program code for carrying out operations of the present disclosure may be written in an object oriented programming language such as Java, Smalltalk, C++ or the like. However, the computer program code for carrying out operations of the present disclosure may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network/a wide area network/the Internet (e.g., network 14).

The present disclosure is described with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, may be implemented by computer program instructions. These computer program instructions may be provided to a processor of a general purpose computer/special purpose computer/other programmable legal research processing apparatus, such that the instructions, which execute via the processor of the computer or other programmable legal research processing apparatus, create means for implementing the functions/acts specified in the flowchart and/or block diagram block or blocks.

These computer program instructions may also be stored in a computer-readable memory that may direct a computer or other programmable legal research processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable legal research processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams in the figures may illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is:

1. A computer-implemented method, executed on a computing device, comprising:
identifying one or more outstanding medical bills associated with one or more patients;
identifying one or more charitable organizations available to at least partially satisfy the one or more outstanding medical bills; and
applying the one or more patients for funding via the one or more charitable organizations.

2. The computer-implemented method of claim 1 wherein the one or more charitable organizations includes one or more of:
a charity;
a non-profit organization and
a philanthropic entity of a for-profit entity.

3. The computer-implemented method of claim 1 wherein the one or more charitable organizations are defined within a charitable organization database.

4. The computer-implemented method of claim 1 wherein the funding includes one or more of:
drug-based funding;
treatment-based funding; and
disease-based funding.

5. The computer-implemented method of claim 1 wherein applying the one or more patients for funding via the one or more charitable organizations includes:
automatically applying the one or more patients for funding via an electronic application process.

6. The computer-implemented method of claim 1 further comprising:
obtaining funding from the one or more charitable organizations for the one or more outstanding medical bills associated with one or more patients.

7. The computer-implemented method of claim 6 further comprising:
applying the obtained funds to at least partially satisfy the one or more outstanding medical bills associated with one or more patients.

8. The computer-implemented method of claim 1 further comprising:
defining a charitable organization database that defines application criteria for a plurality of charitable organizations.

9. The computer-implemented method of claim 1 wherein applying the one or more patients for funding via the one or more charitable organizations includes:
populating an application for the one or more patients for the one or more charitable organizations, thus defining one or more populated applications.

10. The computer-implemented method of claim 7 wherein applying the one or more patients for funding via the one or more charitable organizations further includes:
submitting the one or more populated applications to the one or more charitable organizations.

11. A computer program product residing on a computer readable medium having a plurality of instructions stored thereon which, when executed by a processor, cause the processor to perform operations comprising:
identifying one or more outstanding medical bills associated with one or more patients;
identifying one or more charitable organizations available to at least partially satisfy the one or more outstanding medical bills; and
applying the one or more patients for funding via the one or more charitable organizations.

12. The computer program product of claim 11 wherein the one or more charitable organizations includes one or more of:
a charity;
a non-profit organization and
a philanthropic entity of a for-profit entity.

13. The computer program product of claim 11 wherein the one or more charitable organizations are defined within a charitable organization database.

14. The computer program product of claim 11 wherein the funding includes one or more of:
drug-based funding;
treatment-based funding; and
disease-based funding.

15. The computer program product of claim 11 wherein applying the one or more patients for funding via the one or more charitable organizations includes:
automatically applying the one or more patients for funding via an electronic application process.

16. The computer program product of claim 11 further comprising:
obtaining funding from the one or more charitable organizations for the one or more outstanding medical bills associated with one or more patients.

17. The computer program product of claim 16 further comprising:
applying the obtained funds to at least partially satisfy the one or more outstanding medical bills associated with one or more patients.

18. The computer program product of claim 11 further comprising:
defining a charitable organization database that defines application criteria for a plurality of charitable organizations.

19. The computer program product of claim 11 wherein applying the one or more patients for funding via the one or more charitable organizations includes:
populating an application for the one or more patients for the one or more charitable organizations, thus defining one or more populated applications.

20. The computer program product of claim 17 wherein applying the one or more patients for funding via the one or more charitable organizations further includes:
submitting the one or more populated applications to the one or more charitable organizations.

21. A computing system including a processor and memory configured to perform operations comprising:
identifying one or more outstanding medical bills associated with one or more patients;
identifying one or more charitable organizations available to at least partially satisfy the one or more outstanding medical bills; and
applying the one or more patients for funding via the one or more charitable organizations.

22. The computing system of claim 21 wherein the one or more charitable organizations includes one or more of:
a charity;
a non-profit organization and
a philanthropic entity of a for-profit entity.

23. The computing system of claim 21 wherein the one or more charitable organizations are defined within a charitable organization database.

24. The computing system of claim 21 wherein the funding includes one or more of:
drug-based funding;
treatment-based funding; and
disease-based funding.

25. The computing system of claim 21 wherein applying the one or more patients for funding via the one or more charitable organizations includes:
automatically applying the one or more patients for funding via an electronic application process.

26. The computing system of claim 21 further comprising:
obtaining funding from the one or more charitable organizations for the one or more outstanding medical bills associated with one or more patients.

27. The computing system of claim 26 further comprising:
applying the obtained funds to at least partially satisfy the one or more outstanding medical bills associated with one or more patients.

28. The computing system of claim 21 further comprising:
defining a charitable organization database that defines application criteria for a plurality of charitable organizations.

29. The computing system of claim 21 wherein applying the one or more patients for funding via the one or more charitable organizations includes:
populating an application for the one or more patients for the one or more charitable organizations, thus defining one or more populated applications.

30. The computing system of claim 27 wherein applying the one or more patients for funding via the one or more charitable organizations further includes:
submitting the one or more populated applications to the one or more charitable organizations.
